(19) United States
(12) Patent Application Publication
(10) Pub. No.: US 2022/0288120 A1
(43) Pub. Date: Sep. 15, 2022

(54) METHOD FOR PRODUCING ENGINEERED TIRC7 SPECIFIC T-REGULATORY CELLS (TREGS)

(71) Applicant: Nekonal S.a.r.l., Esch/Alzette (LU)

(72) Inventor: Nalan UTKU, Esch/Alzette (LU)

(21) Appl. No.: 17/635,214

(22) PCT Filed: Aug. 14, 2020
(86) PCT No.: PCT/EP2020/072900

§ 371 Date: Feb. 14, 2022

(30) Foreign Application Priority Data
Aug. 14, 2019 (EP) 19191825.9

(51) Int. Cl. A61K 35/17; C07K 14/725; C07K 16/28

(52) U.S. Cl. CPC A61K 35/17; C07K 14/7051; C07K 16/28; A61K 2039/5158

(57) ABSTRACT

The present invention pertains to an improved composition of cells enriched in regulatory T (Treg) cells and a method for producing the same. In particular, the invention provides a novel strategy to produce such compositions of cells enriched in Tregs, comprising identifying, isolating and expanding cells positive for T cell immune response cDNA 7 (TIRC7) protein as well as CD25. The compositions enriched in Tregs prepared in accordance with the methodology of the invention are particularly useful for medical applications such as the treatment of immunological disorders and infectious diseases. Thus, the present disclosure offers a promising novel treatment approach as a cell-based therapy for patients suffering from immunological disorders, such as allergy, asthma, autoimmune disorders, graft versus host disease (GVHD), and transplantation graft rejection, or infectious diseases.

### FIELD OF THE INVENTION

The invention pertains to an improved composition of cells enriched in regulatory T (Treg) cells and a method for producing the same. In particular, the invention provides a novel strategy to produce such compositions of cells enriched in Tregs, comprising identifying, isolating and expanding cells positive for T cell immune response cDNA 7 (TIRC7) protein as well as CD25. The compositions enriched in Tregs prepared in accordance with the methodology of the invention are particularly useful for medical applications such as the treatment of immunological disorders and infectious diseases. Thus, the present disclosure offers a promising novel treatment approach as a cell-based therapy for patients suffering from immunological disorders, such as allergy, asthma, autoimmune disorders, graft versus host disease (GVHD), and transplantation graft rejection, or infectious diseases.

### DESCRIPTION

A T cell is a type of lymphocyte that plays a central role in cell-mediated immunity. T cells can be distinguished from other lymphocytes, such as B cells and natural killer cells, by the presence of a T-cell receptor on their cell surface. CD4+ T cells are commonly divided into two different subpopulations of T cells, conventional T helper (Th) cells and regulatory T (Treg) cells, formerly known as suppressor T cells. Th cells control adaptive immunity against pathogens by activating other effector immune cells. Treg cells are defined as CD4+ T cells in charge of suppressing potentially deleterious activities of Th cells by downregulating the induction and proliferation of effector T cells. Thus, Tregs, which are present in most tissues, maintain tolerance to self- and non-self antigens and contribute to preventing autoimmune diseases (see, e.g. Fontenot, J. D. & Rudensky, A. Y. Nat Immunol 6, 331-7 (2005); Sakaguchi, S., Annu Rev Immunol 22, 531-62, 2004). Hence, Tregs have clinical applications, e.g. in inflammation, autoimmunity and transplantation, including graft versus host disease (GVHD). Importantly, Tregs have emerged as a promising tool for a cell based clinical application in organ transplantation (e.g. Wood, K. J. & Sakaguchi, S. Nat Rev Immunol 3, 199-210, 2003).

Tregs are known to highly express molecules such as CD3, CD4, CD8, CD25, FOXP3, CTLA4, CD62L, LAP, CD121a, CD121b, LAG-3, GARP, CD103, CD39, Fas, HLA-DR, CD45RO, CD28, ICOS, PD-1, TNFR2, GITR, OX40, 4-1BB, CCR2, CCR4, CCR5, CCR6, CCR7, CCR8, CXCR3, CXCR4, TLR1, TLR2, TLR4, TLR5, TLR6, TLR7, TLR8 and TLR9. Moreover, a low expression of CD127, IFN-gamma, IL10, and CD49d if further characteristic for Tregs (see: Chen et al., 2011, Int Immunopharmacol. 11(10):1489-96; Fu et al., 2004, Am J Transplant. 4(1):65-78).

However, the discrimination of Tregs from effector CD4+ cells is very challenging, and harvesting of sufficient quantities of Tregs relies on the ability to expand Tregs ex vivo. Current protocols for isolation and expansion of Tregs ex vivo produce phenotypically and functionally mixed populations of CD4+ T cells. Moreover, such mixed populations of cells hold risk because they may contain cells with very diverse, sometimes antagonistic functions, which can be able to release pro-inflammatory cytokines.

Thus, there is an unmet need in the art for novel approaches to selectively isolate Tregs as well as expand them without stimulating the expansion of other CD4+ T cell populations, such as effector T cells. Such isolated homogenous human Treg compositions offer the possibility to be applied in adoptive cellular therapy to treat numerous diseases, such as immunological disorders and infectious diseases.

Immunological disorders, such as allergy, asthma, autoimmune disorders, graft versus host disease (GVHD), and transplantation graft rejection, are a major current health problem. Asthma, which is an allergen-induced immunological disorder, is a serious condition, affecting a large proportion of the population (e.g., 8% in the U.S.). Allergies, such as food allergy, seasonal allergy, pet allergy, hives, hay fever, allergic conjunctivitis, poison ivy allergy, oak allergy, mold allergy, drug allergy, dust allergy, cosmetic allergy, and chemical allergy afflict more than 20% of the world's population.

Autoimmune diseases are mediated by the aberrant activation, differentiation and trafficking of leukocytes in response to tissue self-antigens. A number of autoimmune or immune-mediated diseases can be found in humans, such as type I diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis, Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis. Current therapies for such diseases generally involve either the administration of immune suppressants, or the administration of steroids. Such therapies hold the risk of major side effects.

Graft-versus-host disease (GVHD) is a condition that may occur after allogeneic hematopoietic stem cell transplantation (HSCT) and is usually a reaction of donor immune cells against host tissues. GVHD is associated with significant morbidity and mortality. Mortality rates as a direct or indirect consequence of GVHD can reach 50% despite the prophylactic administration of immunosuppressive drugs like cyclosporine, tacrolimus, ATG, and methotrexate.

Furthermore, infectious diseases, such as bacterial infections, viral infections, fungal infections, and parasitic infections, persist as a major worldwide health challenge. The prevalence of pathogens, which are now resistant to previously successful treatments is increasing at an alarming rate (e.g., antibiotic-resistant pathogenic bacteria). Indeed, antimicrobial resistance, largely driven by overuse of antibiotics, is now considered a global health threat.

While the percentage of the affected population and severity of the diseases are rising, current methods for effectively treating immunological disorders and infectious diseases are limited. Thus, there is an urgent and ongoing need to identify new strategies for treating such disorders and diseases. Novel methods to generate a homogenous human Treg composition could enable the use of such compositions in adoptive cellular therapy to treat numerous diseases.

In general, previous attempts to generate human Tregs for adoptive cell therapies are largely susceptible to contamination by non-Tregs, variability in cell function, and the potential for lineage instability following extended periods of in vitro culture. Most importantly, most protocols for isolation of Treg subsets produce cell compositions contaminated with effector T cells. The latter represent an inherent risk of adverse reactions as they drive pro-inflammatory immune reactions by secreting cytokines such as IFN-γ or IL-17.

Accordingly, there is a high risk if such isolated Treg compositions were to be applied in cellular therapy, since it might lead to a potentially fatal activation of the immune system of the patient treated. Such a fatal immune response was recently documented in the failure of the ‘Tegenero’ trials, where an antibody expected to expand Treg cells led to an activation of effector cells (Suntharalingam, G. et al., N Engl J Med 355, 1018-28 (2006)).

WO 2010/022341 describes methods of enriching Tregs as well as the use of such isolated Treg compositions for medicinal applications. In particular, it is identified therein that Tregs may be enriched or isolated based on FOXP3 expression. Furthermore, it is noted that activated regulatory T-cells may be selected based on the presence of at least one of the following molecules: Latency-Associated peptide (LAP), IL-1 receptor type I (CD1 21 a), IL-1 receptor type II (CD1 21 b) and GARP (LRRC32). However, FOXP3 is an intracellular marker molecule, thus, using FOXP3 as a marker for enrichment or purification is not suitable.

Hence, there is an ongoing need for providing improved methods for enrichment, isolation, expansion, and purification of Tregs, while simultaneously maintaining regulatory T cell function. In view of the above described prior art, a continued need in the clinic exists to prepare clinical grade compositions enriched in Tregs, which are suitable for subsequent use in medicinal applications, like prevention or treatment of autoimmune diseases as well as transplant rejection or graft-versus-host diseases by adoptive immunotherapy.

A further object of the invention is to provide composition of cells enriched in Tregs, which are particularly useful in the prevention and treatment of immunological disorders or infectious diseases.

### BRIEF DESCRIPTION OF THE INVENTION

Generally, and by way of brief description, the main aspects of the present invention can be described as follows:

In a first aspect, the invention pertains to an in-vitro method for generating a culture of enriched, preferably human, modified regulatory T-cells (Treg) composition, the method comprising the steps of:
(a) Providing at least one Treg,
(b) Contacting the Treg with at least one TIRC7 specific antigen binding protein (ABP), or a Tirc7 specific antigen binding protein fragment (ABPF) thereof,
(c) Multiplying by culturing the at least one Treg in the presence of the least one TIRC7 specific ABP or ABPF to obtain the enriched modified Treg composition;
(d) Optionally, harvesting the enriched modified Treg composition.

In a second aspect, the invention pertains to an engineered TIRC7 specific T regulatory cell (Treg), comprising a membrane bound TIRC7 specific ABP or ABPF.

In a third aspect, the invention pertains to a TIRC7 specific chimeric antigen receptor (CAR), comprising an ectodomain, a transmembrane domain, and optionally an endodomain, wherein the ectodomain comprises at least one antigen binding site for binding to TIRC7 (SEQ ID NO:2).

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The solution to the above referenced technical problems is achieved in a first aspect of the present invention by using anti-TIRC7 binding agents, optionally together with anti-CD25 binding agents in a process of isolating T regulatory cells. It was surprisingly found that cell populations positive for TIRC7 are enriched in CD4/CD25 positive Tregs. The compositions of cells enriched in Tregs produced in accordance with the methods of the invention are improved Treg-compositions, hence useful in the treatment of various diseases, as will be described herein below.

The term “TIRC7” as used in accordance with the present invention, refers to the T cell immune response cDNA 7 (TIRC7) cell surface protein, which is induced after immune activation in a subset of human T-, B-cells as well as monocytes. TIRC7 has initially been described to be involved in the signal transduction of T-cell activation and proliferation. The soluble form of TIRC7 is capable of inhibiting or suppressing T-cell proliferation in response to alloactivation in a mixed lymphocyte culture or in response to mitogens when exogenously added to the culture. TIRC7 is known to the person skilled in the art and described, inter alia, in WO99/11782, Utku, Immunity 9 (1998), 509-518 and Heinemann, Genomics 57 (1999), 398-406, which also disclose the amino and nucleic acid sequences of TIRC7.

During immune activation, TIRC7 is co-localized with the T cell receptor and CTLA4 within the immune synapse of human T cells. At protein and mRNA level, its expression is induced in lymphocytes in synovial tissues obtained from patients with rheumatoid arthritis or during rejection of solid organ transplants and bone marrow transplantation as well as in brain tissues obtained from patients with multiple sclerosis.

The present invention is based on the surprising finding that Tregs can be isolated based on their expression of TIRC7. Thus, the use of anti-TIRC7 binding agents enables the selection of a composition of cells enriched in Tregs. Accordingly, the present approach represents a hitherto unknown method to selectively isolate Tregs with favorable characteristics. Isolated human Treg compositions can then be applied in adoptive cellular therapy to treat numerous diseases, such as immunological disorders and infectious diseases.

In the first aspect the above mentioned object is solved by a method for producing a composition of cells enriched in T regulatory cells (Tregs), comprising the steps of:
(a) Providing a sample comprising cells from a subject,
(b) Contacting said sample with an anti-TIRC7 binding agent, such as an anti-TIRC7 antibody or ligand, and
(c) separating from said sample a population of cells marked with said anti-TIRC7 binding agent,
   wherein said population of cells marked with said anti-TIRC7 binding agent is a composition of cells enriched in Tregs.

A “binding agent” according to the invention shall refer to a small or large molecule or molecule complex that specifically and (preferably)/or selectively binds to its respective indicated target such as TIRC7 or CD25 protein. Preferred embodiments pertain to antibodies, T cell receptors, or antigen binding fragments thereof, as well as to natural ligands of these molecules. Preferred anti-TIRC7 antibodies that can be used for the present invention are antibodies such as, for example, any of the antibodies disclosed in WO 99/11782, WO 03/054019 and WO 03/054018 (all incorporated herein in their entirety). A preferred antibody is Metiliximab (disclosed in WO 03/054019) as well as any chimerized, humanized or otherwise derivatized variants or fragments thereof, wherein the variant still comprises the CDR1 to CDR3 regions of the parent molecule. Most preferably, the invention pertains to Metiliximab, or any derivatives and chimeras thereof, as an anti-TIRC7 antibody. A TIRC7 ligand is for example HLA class II alpha chain protein or a fragment thereof. Such proteins and their antigen binding fragments are preferred anti-TIRC7 binding agents of the invention. Anti-CD25 binding agents such as antibodies and ligands are well known in the art, and not specifically listed here.

Preferred is the use of monoclonal antibodies and/or fragments thereof (such as Fab or scFv, and the like) for said antigen/antibody reactions that are specific for mammalian, such as human, TIRC7 and/or adducts thereof. If required, the person of skill will be readily able to generate and/or produce suitable antibodies, in particular monoclonal antibodies and/or fragments thereof (such as Fab or scFv, and the like) that are specific for mammalian, such as human, TIRC7 and/or adducts thereof.

The antibodies of the invention are specifically reactive with epitopes on the TIRC7 protein, meaning that they selectively recognize and bind to said epitopes. Any conventional binding assay can be used to assess this binding, including for example, an enzyme linked immunosorbent assay (ELISA).

The antibodies of the invention can be fluorescently labelled, magnetically labelled, biotin labelled or cross-linked. They can further be coupled to beads by any conventional method known in the art.

In some embodiments the problem of the present invention is solved by a method for producing a composition of cells enriched Tregs, wherein said method comprises the steps of providing a sample from a subject, contacting said sample with an anti-TIRC7 antibody and an anti-CD25 antibody, and isolating cells marked with said antibodies from said sample, thereby producing said composition enriched in Tregs.

A sample according to the present disclosure is preferably a biological sample, most preferably a sample comprising a population of cells. Such a sample is for example a tissue or liquid sample of a subject, most preferably wherein said sample is a blood sample or a bone marrow sample from a subject. The population of cells from said sample are, or can include, CD25+ TIRC7+ cells, and/or CD4+CD25+ TIRC7+ cells, which can be isolated or enriched from the blood or bone marrow sample.

A subject according to the present disclosure is a mammal, such as a mouse, rat, guinea pig, rabbit, cat, dog, monkey, or preferably a human.

The method of the present invention can further comprise the step of isolating said cells from a human blood sample or a human bone marrow sample prior to said contacting of the sample with an anti-TIRC7 antibody and an anti-CD25 antibody, and isolating cells marked with said antibodies from said sample, thereby producing said composition enriched in Tregs.

Preferably, the composition of cells obtained by the present invention is a homogeneous population of Tregs with desirable immune modulating properties, e.g. expression of forkhead box P3 (FOXP3) protein. In some embodiments the method may include a step of determining the expression of FOXP3 on the cell comprised in the composition of cells enriched in Tregs.

In a preferred embodiment, the method for producing a composition of cells enriched in Tregs in accordance with the present invention further involves a step of identifying and selecting cells with high expression of at least one further marker, such as, for example, CD3, CD4, CD8, FOXP3, CTLA4, CD62L, LAP, CD121a, CD121b, LAG-3, GARP, CD103, CD39, Fas, HLA-DR, CD45RO, CD28, ICOS, PD-1, TNFR2, GITR, OX40, 4-1BB, CCR2, CCR4, CCR5, CCR6, CCR7, CCR8, CXCR3, CXCR4, TLR1, TLR2, TLR4, TLR5, TLR6, TLR7, TLR8 and TLR9.

In a further preferred embodiment, the method for producing a composition of cells enriched in Tregs according to the present invention further comprises a step of identifying and selecting cells with low expression of at least one further marker, such as, for example, CD127, IFN-gamma, IL10, and CD49d. Such cells may in some embodiments be used for expanding to generate an improved composition of cells enriched Tregs.

For detecting any of the markers indicative for Tregs as named above, commercially available antibodies can be used.

In particular embodiments, said method produces a composition of cells, wherein said Tregs comprise at least 60% of the cells in said composition, preferably at least 70%, more preferably at least 80%, even more preferably at least 90%, most preferably wherein said Tregs comprise substantially 100% of the cells in said composition.

In context of the herein described invention, in all of its embodiments and aspects, it is particularly preferred that the methods of the present invention produces at least 5×10⁶ Tregs, more preferably at least 5×10⁷ Tregs, even more preferably at least 5×10⁸ Tregs, most preferably at least 5×10⁹ Tregs.

Yet another embodiment of the invention pertains to the afore described method for producing a composition of cells enriched in Tregs, the method further comprising a step of either storing said isolated composition of cells enriched in Tregs, or expanding said isolated Tregs.

In addition, the method of producing and expanding a composition enriched in Tregs can include contacting the population of cells with one or more of interleukin-2 (IL2), anti-CD3 (e.g., an anti-CD3 antibody), and/or anti-CD28 (e.g., an anti-CD28 antibody). After in vitro proliferation, the above described method of the invention can produce at least 5×10⁶ Tregs, in which at least 60% of the cells in said composition, preferably at least 70%, more preferably at least 80%, even more preferably at least 90%, most preferably wherein said Tregs comprise substantially 100% of the cells in said composition.

In additional aspects, the invention can include a method for the production of clinical-grade Treg preparations. This method includes the aforementioned method steps for isolating Tregs, but furthermore includes the expansion of the isolated Tregs until receiving an amount of Tregs applicable in the clinic. The expansion of the Tregs of the invention may either follow immediately after isolating the Tregs, or, alternatively, the isolated Tregs are first stored via cryopreservation, and thawing an aliquot of cells prior to starting the expansion process. Methods for the expansion of Tregs are well known to the person of skill.

Another aspect of the invention then pertains to a composition of cells enriched in Tregs obtained by a process comprising the steps of providing a sample from a subject, preferably wherein said sample is a population of cells derived from a tissue of said subject, more preferably wherein said tissue is blood or bone marrow, contacting said sample with an anti-TIRC7 antibody and an anti-CD25 antibody, and isolating cells marked with said antibodies from said sample, thereby producing said composition enriched in Tregs.

A further aspect of the invention then pertains to the use of the composition of cells enriched in Tregs obtained by the method according to the present invention in the treatment of a disease of a subject.

Tregs can generally be used in a wide variety of medical applications. Without intending to be restricted to the following examples, the composition of cells enriched in Tregs of the invention are preferably for use in the treatment of immunological disorders or infectious diseases. However, the composition of cells enriched in Tregs, in accordance with the present invention, can be employed in the treatment, alleviation, or prevention of any disease or disorder.

Another aspect of the present invention pertains to a TIRC7 specific chimeric antigen receptor (CAR), comprising an ectodomain, a transmembrane domain and an optionally an endodomain, wherein the ectodomain comprises at least one binding site for binding to TIRC7 (SEQ ID NO:2).

The term “Chimeric Antigen Receptor” or “CAR” has its general meaning in the art and refers to an artificially constructed hybrid protein or polypeptide containing the antigen binding domains of an antibody (e.g., scFv) linked to a membrane anchor and optionally intracellular signaling domains. Characteristics of CARs include their ability to redirect T-cell or Treg specificity and reactivity toward a selected target in a non-MHC-restricted manner, exploiting the antigen-binding properties of monoclonal antibodies. Strategies to design and produce such CARs are well known in the art, references can be found for example in Bonini and Mondino, Eur. J. Immunol. 2015 (19), Srivastava and Riddell, Trends Immunol. 2015 (20), Jensen and Riddell, Curr. Opin. Immunol. 2015 (21), Gill and June, Immunol. Rev. 2015 (22). In some embodiments, the antigen binding domain comprises a linker peptide. The linker peptide may be positioned between the light chain variable region and the heavy chain variable region.

In some embodiments of the invention the TIRC7 specific CAR comprises an antigen binding site for binding to TIRC7 which is an antibody derived antigen binding site specific for TIRC7, preferably wherein the antibody derived binding site is derived from an anti-TIRC7 antibody that upon binding to TIRC7 reduces T cell activity.

Preferably the antibody derived antigen binding site of the CAR of the invention is at least one antibody variable domain, or antigen binding fragment thereof, preferably is two antibody variable domains. Preferably the antigen binding site of the CAR of the invention comprises at least one antibody heavy chain CDR1 to CDR3 of a source antibody, and at least one antibody light chain CDR1 to CDR3 of the source antibody, preferably wherein the heavy and light chain CDRs are, when expressed, paired with each other to form the antigen binding site. Most preferably the CDR1 to CDR3 sequences are provided in the context of their respective original, or chimerized or humanized variable domain sequences of the source antibody.

In some embodiment of the invention the antibody derived antigen binding site of the TIRC7 specific CAR is an Fab, scFv, or other antibody derived antigen binding fragment, or is an antigen binding site derived from an antibody specific for TIRC7, such as Neliximab or Metiliximab, or is a human antibody Fab or scFv obtained by CDR grafting of the CDR regions of Neliximab or Metiliximab into human variable frameworks.

The antibodies Neliximab and Metiliximab are monoclonal mouse anti-TIRC7 antibodies and disclosed in WO/2003/054019 which is included in its entirety by reference herein. In particular, Neliximab shall be understood as an antibody having the CDR1 to CDR3 of the heavy and light chains (6 CDRs in total) of the antibody disclosed in FIGS. 6 and 7 of WO/2003/054019. Metiliximab, in particular, shall be understood to refer to an antibody having the CDR1 to CDR3 of the heavy and light chains of the antibody disclosed in FIGS. 4 and 5 of WO/2003/054019. The respective sequences shown in these figures shall be also incorporated by reference in the present disclosure.

Hence, in preferred embodiments, a TIRC7 specific CAR is preferred, wherein the ectodomain comprises the 6 CDRs of Neliximab or comprises the 6 CDRs of Metiliximab.

In some embodiments it is preferred that the transmembrane domain of the TIRC7 specific CAR is a CD28 transmembrane domain, or is a membrane anchor.

In some embodiments it is preferred that the endodomain comprises an immune signalling domain, preferably the endodomain comprises a CD3-zeta endodomain with 3 ITAMs. Yet in other embodiments of the invention the endodomain is absent, or is non-functional, which shall be understood to not elicit any signalling events upon binding of the CAR to its target.

In another aspect of the invention there is provided a nucleic acid molecule comprising a sequence encoding the TIRC7 specific CAR according to the herein disclosed invention. The nucleic acid is preferably a vector of any kind, most preferably an expression vector capable of expressing the CAR of the invention, for example in a Treg.

Another aspect then pertains to a recombinant cell, preferably a regulatory T cell (Treg), comprising the TIRC7 specific CAR or a nucleic acid according to the invention.

Yet another aspect of the invention then pertains to a method for generating an engineered regulatory T-cell (Treg), the method comprising the steps of:
(a) Providing at least one Treg,
(b) Introducing into said Treg a first genetic expression construct for the expression of a first TIRC7 specific antigen binding protein (ABP), or a TIRC7 specific antigen binding protein fragment (ABPF) thereof,
(c) Expressing said first genetic expression construct in said Treg to obtain a modified Treg.

In a preferred embodiments of the method said Treg is derived from a composition of cells produced according to a Treg preparation method disclosed herein above, and/or wherein said Treg is derived from a composition of cells disclosed herein as part of the invention.

In some preferred embodiments said Treg is a Treg isolated from a patient suffering from a disorder, preferably wherein the disorder is treatable by modulation of the activity of Treg in the patient, such as a cancer or immune disorder. The diseases mentioned are in detail described herein elsewhere. Preferred is the use of such CAR and Treg in autoimmune disorders.

The method according to the invention in some embodiments, further comprises:
Multiplying by culturing said modified Treg to obtain an enriched modified Treg composition.

In some preferred embodiments, the TIRC7 specific ABP, or said TIRC7 specific ABPF, specifically binds an extracellular domain, or the C-terminus of TIRC7, preferably the ABP or ABPF specifically (i) binds the extracellular domain of TIRC7 between the 1^{st} and the 2^{nd} transmembrane domain, or (ii) binds the extracellular domain of TIRC7 between the 3^{st} and the 4^{th} transmembrane domain, or (iii) binds the extracellular domain of TIRC7 between the 5^{th} and the 6^{th} transmembrane domain, or (iv) binds the c-terminal region c-terminal of the 7^{th} transmembrane domain, wherein the numbering of the transmembrane domains is in the direction of N to C terminus. TIRC7 domain structure is provided in FIG. 5.

In preferred embodiments of the method of the invention step (b) further comprises introducing into said Treg a second genetic expression construct for the expression of a second TIRC7 specific ABP, or TIRC7 specific ABPF thereof, wherein said second TIRC7 specific ABP does not equal the first TIRC7 specific ABP, and preferably wherein the first and the second TIRC7 specific ABP, and their respective TIRC7 specific ABPFs, bind to different TIRC7 epitopes. By using a combination of TIRC7 specific ABPs and in particular CARs, the immunomodulatory potential of the Treg of the invention can be increased significantly.

Preferably, the first and the second TIRC7 specific ABP, specifically bind different extracellular regions of TIRC7, preferably different regions as defined in item B 34, numbers (i) to (vi), for example wherein the first TIRC7 specific ABP binds to the C-terminal region c-terminal of the 7^{th} transmembrane domain, and the second ABP specifically binds TIRC7 at an extracellular domain (i) to (iii).

In further preferred embodiments of the method of the invention step (b) further comprises introducing into said Treg at least one additional genetic expression construct for the expression of at least one further (3^{rd}, 4^{th}, 5^{th}, 6^{th} etc.) TIRC7 specific ABP, or TIRC7 specific ABPF thereof, wherein said at least one further TIRC7 specific ABP does not equal the first or second TIRC7 specific ABP.

Yet further embodiments are preferred wherein at least one ABP or ABPF is selected from a T cell receptor, or a fragment thereof, or an antibody, or a fragment thereof. Most preferably the ABP or ABPF is an extracellular protein, and preferably located to the cell membrane, for example, the ABP or ABPF comprises at least one transmembrane domain, or other membrane anchor.

For example, and preferably, the ABP or ABPF comprises at least one antigen binding site derived from an antibody or T cell receptor, and preferable comprises an antigen binding fragment of an antibody such as an antibody Fab, or antibody scFv.

The method of the invention is preferred, wherein the TIRC7 specific ABP, or ABPF, binds to an extracellular sequence of TIRC7 selected from:
amino acids 210-228 of SEQ ID NO:2;
amino acids 347-355 of SEQ ID NO:2;
amino acids 438-512 of SEQ ID NO:2; or
amino acids 586-614 of SEQ ID NO:2.

These regions in TIRC7 are shown for example in FIG. 5.

The TIRC7 specific ABP is preferably an antibody, or antigen binding fragment thereof, selected from Neliximab or Metiliximab, or CDR grafted humanized versions thereof, also described above.

In another embodiment of the invention, the ABPF or fragments of an antibody, are preferably proteins comprising at least an antibody or TCR variable region, preferably two, such as a Fab or scFv fragment.

The TIRC7 specific ABP is most preferably a chimeric antigen receptor (CAR), and wherein the obtained Treg is a CAR-Treg.

Another aspect of the invention then pertains to an engineered TIRC7 specific T regulatory cell (Treg), comprising a membrane bound TIRC7 specific antigen binding protein (ABP), or a TIRC7 specific antigen binding protein fragment (ABPF). Preferably the TIRC7 specific ABP, or TIRC7 specific ABPF, is an ABP or ABPF as defined according to the present invention.

The engineered TIRC7 specific Treg according to the invention is preferred wherein it expresses a chimeric antigen receptor (CAR) comprising in that order an (i) extracellular domain comprising a TIRC7 specific antigen binding site, (ii) a membrane domain or membrane anchor, and (iii) an intracellular domain comprising a signalling domain. Preferably, the CAR is an TIRC7 specific CAR as described herein above. Most preferably, the engineered Treg comprises more than one, preferably two, three, four or more TIRC7-specific CAR according to the invention, wherein the multiple TIRC7-specific CAR are specific to distinct and non-identical extracellular epitopes of TIRC7.

In accordance with the herein described aspects and embodiments of the invention a disease preferably is an immunological disorder or infectious disease.

In particular embodiments, said immunological disorders are selected from the group consisting of allergy, asthma, an autoimmune disorder, graft versus host disease (GVHD), and transplantation graft rejection.

Allergies treatable by the composition and methods of the invention include food allergy, seasonal allergy, pet allergy, hives, hay fever, allergic conjunctivitis, poison ivy allergy, oak allergy, mold allergy, drug allergy, dust allergy, cosmetic allergy, and chemical allergy.

In context of the herein disclosed invention, the term “autoimmune disorders” shall include the following disorders, which may each be a disorder preferred for the herein disclosed invention: type I diabetes, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis.

An infectious disease of the invention is preferably selected from the group consisting of a bacterial infection, a viral infection, a fungal infection, and a parasitic infection.

In some aspects of the invention, the invention provides a method of treating a subject in need thereof comprising the step of administering a therapeutically effective amount of the composition of cells enriched in Tregs produced in accordance with the present invention. The composition enriched in Tregs can be produced by any method known in the art. One method of producing a composition enriched in Tregs is by using the methods of the invention described above.

Another aspect of the invention pertains further to a method of treating a disease in a subject, wherein the treatment comprises obtaining a cellular sample from a subject, producing with said sample a composition of cells enriched in T regulatory cells (Tregs) according to the inventive method as described herein before, optionally purifying the so produced composition of cells enriched in Tregs, and administering said (optionally purified) composition of cells enriched in Tregs to a subject in need of the treatment.

In some embodiments the method further comprises a step of expanding Tregs in the composition of cells enriched in Tregs before administering said composition of cells enriched in Tregs to the subject.

The treatment in some embodiments is an autologous cell therapy wherein said sample obtained from a subject is a sample from the subject in need of the treatment (autologous treatment).

In embodiments where the treatment is an autologous cell therapy, it may be preferable that said sample obtained from a subject in need of a treatment is obtained at an earlier time point from the (same) subject, for example at a time point the subject has not yet developed the disease (where the subject is a subject at risk of developing the disease), or is in an early stage of the disease, or is in remission or was successfully treated—for example by a first line treatment—, or suffers from the disease but has a generally stable state of health, such that obtaining the sample from the subject does not put the life and/or health of the subject at risk. In this embodiment it is preferred that the so produced composition of cells enriched in Tregs derived from said sample is afterwards stored for later use. Thus, the administration of the composition of cells enriched in Tregs to the subject in need of the treatment will be performed at a later time point when the subject is in immediate need of the treatment. For example, the later time point when the subject is in need of the treatment could be when the subject develops the disease for the first time, or when the subject suffers from a deterioration of the disease, or when the subject suffers from a relapse or complication of the disease, for example after or during a successful first line therapy. This embodiment therefore allows a preventive generation and storing of Treg compositions for autologous cell therapy as described herein before. This is in particular applicable in scenarios where the subject is known to be at risk of developing the disease or a deterioration of the disease in the future.

The treatment in some other embodiments is a heterologous cell therapy wherein said sample obtained from a subject is a sample not from the subject in need of the treatment, such as a healthy subject, and/or genetically closely related subject, such as sibling (heterologous treatment).

In another aspect there is provided an anti-TIRC7 binding agent as described before, for use in the treatment methods of the invention.

A therapeutically effective amount of the composition for use in the treatment of any of the diseases named above includes at least 5×10⁶ (e.g., 5×10⁶, 5×10⁷, 5×10⁸, 5×10⁹, 5×10¹⁰, 5×10¹¹, or 5×10¹²) Tregs, which are administered to a patient suffering from an immunological disorder or infectious disease.

Given the composition of cells enriched in Tregs of the present invention, the number of cells to be administered is dependent upon a variety of factors, including the age, weight, and sex of the patient, the disease to be treated, and the extent and severity thereof.

The composition of cells enriched in Tregs of the invention can be administered by a variety of procedures, e.g. systemically, such as by intravenous, intra-arterial, or intraperitoneal administration. The cells can be administered by direct injection to an organ or tissue in need thereof, e.g. during a surgical procedure. The composition of cells enriched in Tregs can also be applied topically.

A cell therapy involving administration of a composition of cells enriched in Tregs is based, for example, on the following steps: produce a composition of cells enriched in Tregs according to the methods of the present invention, isolate and expand Tregs in accordance with the herein described methods, and administer the composition of cells enriched in Tregs to the subject/patient, with or without biochemical or genetic manipulation.

Another aspect of the invention is a method for the production of a medicament comprising a composition of cells enriched in Tregs, comprising the method steps according to any of the herein described methods for the isolation/purification of Tregs.

In some embodiments, the herein disclosed methods are ex vivo or in vitro methods, preferably wherein the methods are conducted completely ex vivo or in vitro.

Yet another aspect of the present invention then relates to a pharmaceutical composition comprising the composition of cells enriched in Tregs obtained by the method as described above, or the composition of cells enriched in Tregs for use in the treatment of a disease of a subject; and a pharmaceutically acceptable carrier, stabilizer and/or excipient.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer a composition of cells according to the invention by certain routes of administration, it may be necessary to co-administer the cells with a material to prevent their inactivation or activation. For example, the composition of cells may be administered to a subject in an appropriate carrier, for example, liposomes, or a diluent. Pharmaceutically acceptable diluents include saline and aqueous buffer solutions. A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, in one embodiment the carrier is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. An “immunoconjugate” is an antibody conjugated to one or more heterologous molecule(s), including but not limited to a cytotoxic agent.

The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously; the pharmaceutical composition may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

In yet another aspect of the invention, the invention provides a kit for conducting the method of the invention, comprising one or more of the following: culture medium, and instructions for using same. In another embodiment the invention can provide a kit comprising a sample of the composition of cells enriched in Tregs obtained by the process as described by the present invention and optionally culture medium and or instructions for use in experiments and/or in transplantation.

In context of the present invention the term “subject”, as used in certain embodiments, preferably refers to a mammal, such as a mouse, rat, guinea pig, rabbit, cat, dog, monkey, or preferably a human. The term “patient” preferably refers to a mammal, such as a mouse, rat, guinea pig, rabbit, cat, dog, monkey, or preferably a human, for example a human patient. The subject of the invention may be at danger of suffering from an immunological disorder (such as an allergy, asthma, an autoimmune disorder, graft versus host disease (GVHD), and transplantation graft rejection) or an infectious disease (such as bacterial infection, a viral infection, a fungal infection, and a parasitic infection). A more detailed description of medical indications relevant in context of the invention is provided herein elsewhere.

For the purpose of the present invention, the term “antibody”, as used herein, includes whole antibodies or immunoglobulins and any antigen-binding fragments or single chains thereof. Antibodies, as used herein, can be mammalian (e.g., human or mouse), humanized, chimeric, recombinant, synthetically produced, or naturally isolated. In most mammals, including humans, whole antibodies have at least two heavy (H) chains and two light (L) chains connected by disulfide bonds. Each heavy chain consists of a heavy chain variable region (abbreviated herein as VH) and a heavy chain constant region. The heavy chain constant region consists of three domains, CH1, CH2, and CH3 and a hinge region between CH1 and CH2. Each light chain consists of a light chain variable region (abbreviated herein as VL) and a light chain constant region. The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to factors in the host tissue, including various cells of the immune system (e.g., effector cells) and the first component (Clq) of the classical complement system. Antibodies of the present invention include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

The term “antigen-binding fragment,” as used herein, refers to one or more fragments of an antibody that retain the ability to specifically bind to a specific antigen (e.g., CD21 receptor). The antigen-binding function of an antibody can be performed by fragments of a full-length antibody. The antibody fragments can be a Fab, Fab′2, scFv, SM IP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V|_, VH, C|_, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment (Ward et al., Nature 341:544-546, 1989), which consists of a VH domain; (vii) a dAb which consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al., Science 242: 423-426, 1988, and Huston et al., Proc. Natl. Acad. Sci. USA 85: 5879-5883, 1988). These antibody fragments are obtained using conventional techniques known to those of skill in the art, and the fragments are screened for utility in the same manner as intact antibodies. Antigen-binding fragments can be produced by recombinant DNA techniques or by enzymatic or chemical cleavage of intact immunoglobulins.

Preferred antigen binding proteins (ABP) of the invention comprise an antigen binding domain derived from the prior art TIRC7 antibodies neliximab or metiliximab. Those antibodies are disclosed in EP 1 492 817 (A2), in particular the variable domain sequences are for example shown in FIGS. 4 to 7 of EP1492817 which are included herein by reference. An antigen binding domain of an ABP of the invention is therefore preferably characterized to be an anti-TIRC7 antibody or antigen binding fragment thereof comprising all three complementarity determining regions (CDRs) of the VH and all three complementarity determining regions (CDRs) of the VL variable regions, wherein the amino acid sequences of said VH and VL variable regions are set forth in (i) FIGS. 4 and 5 of EP1492817 for metiliximab (said sequence of the figure of EP1492817 incorporated herein by reference), or (ii) FIGS. 6 and 7 of EP1492817 for neliximab (said sequence of the figure of EP1492817 incorporated herein by reference).

The term “chimeric antibody” refers to an immunoglobulin or antibody whose variable regions derive from a first species and whose constant regions derive from a second species. Chimeric antibodies can be constructed, for example, by genetic engineering, from immunoglobulin gene segments belonging to different species (e.g., from a mouse and a human).

The term “human antibody” as used herein, is intended to include antibodies, or fragments thereof, having variable regions in which both the framework and CDR regions are derived from human germline immunoglobulin sequences as described, for example, by Kabat et al (Sequences of proteins of Immunological Interest, Fifth Edition, U.S. Department of Health and Human Services, NIH Publication No. 91-3242, 1991). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (i.e., a humanized antibody or antibody fragment).

The term “humanized antibody” refers to any antibody or antibody fragment that includes at least one immunoglobulin domain having a variable region that includes a variable framework region substantially derived from a human immunoglobulin or antibody and complementarity determining regions (e.g., at least one CDR) substantially derived from a non-human immunoglobulin or antibody.

The term “substantially 100%” or “substantially homogeneous” as used herein with respect to a Treg enriched composition of the invention means at least 90%, 95%, 96%, 97%, 98%, or 99% or more (e.g., all) of the cells in the composition are Tregs.

The term “treating” as used herein means stabilizing or reducing an adverse symptom associated with a condition; reducing the severity of a disease symptom; slowing the rate of the progression of a disease; inhibiting or stabilizing the progression of a disease condition; or changing a metric that is associated with the disease state in a desirable way.

Furthermore, the invention is defined by the following itemized embodiments:

Item 1. A method for producing a composition of cells enriched in T regulatory cells (Tregs), comprising the steps of:
a) Providing a sample comprising cells from a subject,
b) Contacting said sample with an anti-TIRC7 binding agent, such as an anti-TIRC7 antibody or ligand, or peptide or soluble protein and
c) separating from said sample a population of cells marked with said anti-TIRC7 binding agent,
wherein said population of cells marked with said anti-TIRC7 binding agent is a composition of cells enriched in Tregs.
Item 2. The method according to item 1, wherein said sample is a liquid or a tissue sample of said subject; preferably a blood or bone marrow sample.
Item 3. The method according to item 1 or 2, wherein said subject is a mammal, such as a mouse, rat, guinea pig, rabbit, cat, dog, monkey, and preferably is a human.
Item 4. The method according to any one of items 1 to 3, wherein step b) further comprises contacting said sample with an anti-CD25 binding agent, such as an CD25 antibody or ligand; and wherein step c) comprises separating from said sample a population of cells marked with said anti-TIRC7 and anti-CD25 binding agent.
Item 5. The method according to any one of items 1 to 4, further comprising the step of identifying and selecting cells from said population of cells with high expression of at least one, preferably two, three, four or more, further marker, such as, for example, CD3, CD4, CD8, FOXP3, CTLA4, CD62L, LAP, CD121a, CD121b, LAG-3, GARP, CD103, CD39, Fas, HLA-DR, CD45RO, CD28, ICOS, PD-1, PDL1, TNFR2, GITR, OX40, 4-1BB, CCR2, CCR4, CCR5, CCR6, CCR7, CCR8, CXCR3, CXCR4, TLR1, TLR2, TLR4, TLR5, TLR6, TLR7, TLR8 and/or TLR9.
Item 6. The method according to any one of items 1 to 5, further comprising the step of identifying and selecting cells from said population of cells with expression of at least one further marker, such as, for example, foxp3, CD127, IFN-gamma, IL10, and/or CD49d.
Item 7. The method according to any of items 1 to 6, wherein in said population of cells Tregs comprise at least 60% of the cells in said composition, preferably at least 70%, more preferably at least 80%, even more preferably at least 90%, most preferably wherein said Tregs comprise substantially 100% of the cells in said composition.
Item 8. The method according to any one of items 1 to 6, wherein said method produces at least 2×10⁶ Tregs, more preferably at least 5×10⁷ Tregs, even more preferably at least 5×10⁸ Tregs, most preferably more than 5×10⁹ Tregs.
Item 9. The method according to any one of items 1 to 8, further comprising a step of removing said binding agent or binding agents from said composition of cells enriched in Tregs.
Item 10. The method according to any one of items 1 to 9, further comprising a step of storing said composition of cells enriched in Tregs.
Item 11. The method according to any one of items 1 to 10, further comprising a step of expanding Tregs from said composition of cells enriched in Tregs.
Item 12. The method according to any one of items 1 to 11, wherein a population of cells marked with said anti-TIRC7 binding agent, optionally additionally marked with said anti-CD25 binding agent, is characterized by a specific and selective binding between said anti-TIRC7 binding agent, optionally said anti-CD25 binding agent, and said cells.
Item 13. The method according to any one of items 1 to 12, wherein step b) comprises a contacting of said binding agent with said sample under conditions which allow for a specific and/or selective binding of the binding agent to its target.
Item 14. A composition of cells enriched in T regulatory cells (Tregs) obtainable by a method according to any one of items 1 to 13.
Item 15. A composition of cells according to item 14 for use in the treatment of a disease of a subject.
Item 16. The composition of cells for use according to item 15, wherein said disease is a proliferative disease, immunological disorder or an infectious disease.
Item 17. The composition of cells for use according to item 16, wherein said immunological disorder is selected from the group consisting of allergy, asthma, an autoimmune disorder, graft versus host disease (GVHD), and transplantation graft rejection.
Item 18. The composition of cells for use according to item 17, wherein said allergy is selected from the group consisting of food allergy, seasonal allergy, pet allergy, hives, hay fever, allergic conjunctivitis, poison ivy allergy, oak allergy, mold allergy, drug allergy, dust allergy, cosmetic allergy, and chemical allergy.
Item 19. The composition of cells for use according to item 17, wherein said autoimmune disorder is selected from the group consisting of type I diabetes, Alopecia Areata, Ankylosing Spondylitis, Antiphospholipid Syndrome, Autoimmune Addison's Disease, Autoimmune Hemolytic Anemia, Autoimmune Hepatitis, Primary sclerosing Cholangitis, TIRC7+ inflammatory Stage of Intra Hepatic Cholangiocarcinoma, Gastro-intestinal and generalized IgG4 autoimmune diseases, Atopic Dermatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Chronic Fatigue Syndrome, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis, Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, and Wegener's Granulomatosis.
Item 20. The composition of cells for use according to item 15, wherein said infectious disease is selected from the group consisting of a bacterial infection, a viral infection, a fungal infection, and a parasitic infection.
Item 21. A pharmaceutical composition comprising the composition of cells according to item 14; and a pharmaceutically acceptable carrier, stabilizer and/or excipient.
Item 22. An anti-TIRC7 binding agent for use in the treatment of a disease, wherein the treatment comprises obtaining a cellular sample from a subject, producing with said sample a composition of cells enriched in T regulatory cells (Tregs) according to any one of items 1 to 13, optionally purifying the so produced composition of cells enriched in Tregs, and administering said (optionally purified) composition of cells enriched in Tregs to a subject in need of the treatment.
Item 23. The anti-TIRC7 binding agent for use according to item 22, wherein the anti-TIRC7 binding agent is selected from an antibody, or an antigen binding fragment thereof, preferably neliximab or metiliximab, or fully human anti-TIRC7 antibody or humanized anti-TIRC7 antibody, antigen binding fragments thereof (as described in WO 99/11782, WO 03/054018 or WO 03/054019), or is a TIRC7 ligand such as HLA class II alpha chain protein or a fragment thereof.
Item 24. The anti-TIRC7 binding agent for use according to item 22 or 23, wherein said sample is a tissue or a liquid sample, and preferably is a blood or bone marrow sample.
Item 25. The anti-TIRC7 binding agent for use according to any one of items 22 to 24, further comprising a step of expanding Tregs in the composition of cells enriched in Tregs before administering said composition of cells enriched in Tregs to the subject.
Item 26. The anti-TIRC7 binding agent for use according to any one of items 22 to 25, wherein said sample obtained from a subject is a sample from the subject in need of the treatment (autologous treatment).
Item 27. The anti-TIRC7 binding agent for use according to item 26, wherein said sample obtained from a subject in need of a treatment is obtained at a time point the subject has not yet developed the disease, or is at an early stage of the disease, or is in remission or was successfully treated, or suffers from the disease but has a generally stable state of health; and wherein the so produced composition of cells enriched in Tregs is then stored and later administered to the subject in need of the treatment at a time point the subject is in need of the treatment.
Item 28. The anti-TIRC7 binding agent for use according to item 27, wherein the time point the subject is in need of the treatment is when the subject gets the disease, or when the subject suffers from a deterioration of the disease, or when the subject suffers from a relapse of the disease.
Item 29. The anti-TIRC7 binding agent for use according to any one of items 22 to 25, wherein said sample obtained from a subject is a sample not from the subject in need of the treatment, such as a healthy subject, and/or genetically closely related subject, such as sibling (heterologous treatment).
Item 30. A method for generating a modified T-regulatory cell (Treg), the method comprising the steps of:
(a) Providing at least one Treg,
(b) Introducing into said Treg a first genetic expression construct for the expression of a first TIRC7 specific antigen binding protein (ABP), or a TIRC7 specific antigen binding protein fragment (ABPF) thereof,
(c) Expressing said first genetic expression construct in said Treg to obtain a modified Treg.
Item 31. The method according to item 30, wherein said Treg is derived from a composition of cells produced according to a method of any one of the preceding items, and/or wherein said Treg is derived from a composition of cells of any one of the preceding items.
Item 32. The method according to item 30, wherein said Treg is a Treg isolated from a patient suffering from a disorder, preferably wherein the disorder is treatable by modulation of the activity of Treg in the patient, such as a cancer or immune disorder.
Item 33. The method according to any one of items 30 to 32, further comprising: (d) Multiplying by culturing said modified Treg to obtain an enriched modified Treg composition.
Item 34. The method according to any one of items 30 to 33, wherein said TIRC7 specific ABP, or said TIRC7 specific ABPF, specifically binds an extracellular domain, or the C-terminus of TIRC7, preferably the ABP or ABPF specifically (i) binds the extracellular domain of TIRC7 between the 1^{st} and the 2^{nd} transmembrane domain, or (ii) binds the extracellular domain of TIRC7 between the 3^{st} and the 4^{th} transmembrane domain, or (iii) binds the extracellular domain of TIRC7 between the 5^{th} and the 6^{th} transmembrane domain, or (iv) binds the c-terminal region c-terminal of the 7^{th} transmembrane domain, wherein the numbering of the transmembrane domains is in the direction of N to C terminus.
Item 35. The method according to any one of items 30 to 34, wherein the step (b) further comprises introducing into said Treg a second genetic expression construct for the expression of a second TIRC7 specific ABP, or TIRC7 specific ABPF thereof, wherein said second TIRC7 specific ABP does not equal the first TIRC7 specific ABP, and preferably wherein the first and the second TIRC7 specific ABP, and their respective TIRC7 specific ABPFs, bind to different TIRC7 epitopes.
Item 36. The method according to item 35, wherein the first and the second TIRC7 specific ABP, specifically bind different extracellular regions of TIRC7, preferably different regions as defined in item 34, numbers (i) to (vi), for example wherein the first TIRC7 specific ABP binds to the C-terminal region c-terminal of the 7^{th} transmembrane domain, and the second ABP specifically binds TIRC7 at an extracellular domain (i) to (iii).
Item 37. The method according to any one of items 35 to 36, wherein the step (b) further comprises introducing into said Treg at least one additional genetic expression construct for the expression of at least one further (3^{rd}, 4^{th}, 5^{th}, 6^{th} etc.) TIRC7 specific ABP, or TIRC7 specific ABPF thereof, wherein said at least one further TIRC7 specific ABP does not equal the first or second TIRC7 specific ABP.
Item 38. The method according to any one of items 30 to 37, wherein at least one ABP is selected from a T cell receptor, or a fragment thereof, or an antibody, or a fragment thereof.
Item 39. The method according to any one of items 30 to 38, wherein the ABP or ABPF is an extracellular protein, and preferably located to the cell membrane, for example, the ABP or ABPF comprises at least one transmembrane domain, or other membrane anchor.
Item 40. The method according to any one of items 30 to 39, wherein the ABP or ABPF comprises at least one antigen binding site derived from an antibody or T cell receptor, and preferable comprises an antibody Fab, or antibody scFv [ . . . ]
Item 41. A method according to any one of items 30 to, wherein the TIRC7 specific ABP, or ABPF, binds to an extracellular sequence of TIRC7 selected from:
(d) amino acids 210-228 of SEQ ID NO:2;
(e) amino acids 347-355 of SEQ ID NO:2;
(f) amino acids 438-512 of SEQ ID NO:2; or
(g) amino acids 586-614 of SEQ ID NO:2.
Item 42. The method according to any one of items 30 to 41, wherein the TIRC7 specific ABP us an antibody, or antigen binding fragment thereof, selected from Neliximab or Metiliximab, or humanized mAb thereof.
Item 43. The method according to any one of items 30 to 42, wherein an ABPF or fragments of an antibody, are preferably proteins comprising at least an antibody or TCR variable region, preferably two, such as an Fab or scFv fragment.
Item 44. The method according to any one of items 30 to 43, wherein the TIRC7 specific ABP is a chimeric antigen receptor (CAR), and wherein the obtain Treg is an CAR-Treg.
Item 45. An engineered TIRC7 specific T regulatory cell (Treg), comprising a membrane bound TIRC7 specific antigen binding protein (ABP), or a TIRC7 specific antigen binding protein fragment (ABPF).
Item 46. The engineered TIRC7 specific Treg according to item 45, wherein the TIRC7 specific ABP, or TIRC7 specific ABPF, is an ABP or ABPF as defined in any of items 30 to 44.
Item 47. The engineered TIRC7 specific Treg according to any one of items 45 to 46, which expresses a chimeric antigen receptor (CAR) comprising in that order an (i) extracellular domain comprising a TIRC7 specific antigen binding site, (ii) a membrane domain or membrane anchor, and (iii) an intracellular domain comprising a signalling domain.

Furthermore, the invention is also defined by the following itemized B-embodiments:

Item B1: A TIRC7 specific chimeric antigen receptor (CAR), comprising an ectodomain, a transmembrane domain and an optionally an endodomain, wherein the ectodomain comprises at least one binding site for binding to TIRC7 (SEQ ID NO:2).
Item B2: The TIRC7 specific CAR according to item B 1, wherein the binding site for binding to TIRC7 is an antibody derived antigen binding site specific for TIRC7, preferably wherein the antibody derived binding site is derived from an anti-TIRC7 antibody that upon binding to TIRC7 reduces T cell activity.
Item B3: The TIRC7 specific CAR according to item B 2, wherein the antibody derived antigen binding site is at least one antibody variable domain, or antigen binding fragment thereof, preferably is two antibody variable domains.
Item B4: The TIRC7 specific CAR according to any of item Bs 2 or 3, wherein the antibody derived antigen binding site specific for TIRC7 is an Fab, scFv, or antigen binding site derived from an antibody specific for TIRC7, such as Neliximab or Metiliximab, or is a human or humanized antibody versions thereof, Fab or scFv obtained by CDR grafting of the CDR regions of Neliximab or Metiliximab into human variable frameworks.
Item B5: The TIRC7 specific CAR according to any one of item Bs 1 to 4, wherein the ectodomain comprises the 6 CDRs of Neliximab or comprises the 6 CDRs of Metiliximab.
Item B6: The TIRC7 specific CAR according to any one of item Bs 1 to 4, wherein the transmembrane domain is CD28 transmembrane domain, or is a membrane anchor.
Item B7: The TIRC7 specific CAR according to any one of item Bs 1 to 6, wherein the endodomain comprises an immune signalling domain, preferably the endodomain comprises a CD3-zeta endodomain with 3 ITAMs.
Item B8: A nucleic acid molecule comprising a sequence encoding the TIRC7 specific CAR according to any one of the preceding item Bs.
Item B9: A recombinant cell, preferably a regulatory T cell (Treg), comprising the TIRC7 specific CAR or a nucleic acid according to any one of the preceding item Bs.
Item B10: A method for generating an engineered regulatory T-cell (Treg), the method comprising the steps of:
(a) Providing at least one Treg,
(b) Introducing into said Treg a first genetic expression construct for the expression of a first TIRC7 specific antigen binding protein (ABP), or a TIRC7 specific antigen binding protein fragment (ABPF) thereof,
(c) Expressing said first genetic expression construct in said Treg to obtain a modified Treg.
Item B11: The method according to item B 10, wherein said Treg is derived from a composition of cells produced according to a method of any one of the preceding item Bs, and/or wherein said Treg is derived from a composition of cells of any one of the preceding item Bs.
Item B12: The method according to item B 11, wherein said Treg is a Treg isolated from a patient suffering from a disorder, preferably wherein the disorder is treatable by modulation of the activity of Treg in the patient, such as a cancer or immune disorder.
Item B13: The method according to any one of item Bs 10 to 12, further comprising: (d) Multiplying by culturing said modified Treg to obtain an enriched modified Treg composition.
Item B14: The method according to any one of item Bs 10 to 13, wherein said TIRC7 specific ABP, or said TIRC7 specific ABPF, specifically binds an extracellular domain, or the C-terminus of TIRC7, preferably the ABP or ABPF specifically (i) binds the extracellular domain of TIRC7 between the 1^{st} and the 2^{nd} transmembrane domain, or (ii) binds the extracellular domain of TIRC7 between the 3^{st} and the 4^{th} transmembrane domain, or (iii) binds the extracellular domain of TIRC7 between the 5^{th} and the 6^{th} transmembrane domain, or (iv) binds the c-terminal region c-terminal of the 7^{th} transmembrane domain, wherein the numbering of the transmembrane domains is in the direction of N to C terminus.
Item B15: The method according to any one of item Bs 10 to 14, wherein the step (b) further comprises introducing into said Treg a second genetic expression construct for the expression of a second TIRC7 specific ABP, or TIRC7 specific ABPF thereof, wherein said second TIRC7 specific ABP does not equal the first TIRC7 specific ABP, and preferably wherein the first and the second TIRC7 specific ABP, and their respective TIRC7 specific ABPFs, bind to different TIRC7 epitopes.
Item B16: The method according to item B 15, wherein the first and the second TIRC7 specific ABP, specifically bind different extracellular regions of TIRC7, preferably different regions as defined in item B 34, numbers (i) to (vi), for example wherein the first TIRC7 specific ABP binds to the C-terminal region c-terminal of the 7^{th} transmembrane domain, and the second ABP specifically binds TIRC7 at an extracellular domain (i) to (iii).
Item B17: The method according to any one of item Bs 14 to 16, wherein the step (b) further comprises introducing into said Treg at least one additional genetic expression construct for the expression of at least one further (3^{rd}, 4^{th}, 5^{th}, 6^{th} etc.) TIRC7 specific ABP, or TIRC7 specific ABPF thereof, wherein said at least one further TIRC7 specific ABP does not equal the first or second TIRC7 specific ABP.
Item B18: The method according to any one of item Bs 10 to 17, wherein at least one ABP or ABPF is selected from a T cell receptor, or a fragment thereof, or an antibody, or a fragment thereof.
Item B19: The method according to any one of item Bs 10 to 18, wherein the ABP or ABPF is an extracellular protein, and preferably located to the cell membrane, for example, the ABP or ABPF comprises at least one transmembrane domain, or other membrane anchor.
Item B20: The method according to any one of item Bs 10 to 19, wherein the ABP or ABPF comprises at least one antigen binding site derived from an antibody or T cell receptor, and preferable comprises an antigen binding fragment of an antibody such as an antibody Fab, or antibody scFv.
Item B21: A method according to any one of item Bs 10 to 20, wherein the TIRC7 specific ABP, or ABPF, binds to an extracellular sequence of TIRC7 selected from:

amino acids 210-228 of SEQ ID NO:2;

amino acids 347-355 of SEQ ID NO:2;

amino acids 438-512 of SEQ ID NO:2; or

amino acids 586-614 of SEQ ID NO:2.

Item B22: The method according to any one of item Bs 10 to 21, wherein the TIRC7 specific ABP us an antibody, or antigen binding fragment thereof, selected from Neliximab or Metiliximab, or CDR grafted humanized versions thereof.
Item B23: The method according to any one of item Bs 10 to 22, wherein an ABPF or fragments of an antibody, are preferably proteins comprising at least an antibody or TCR variable region, preferably two, such as an Fab or scFv fragment.
Item B24: The method according to any one of item Bs 10 to 23, wherein the TIRC7 specific ABP is a chimeric antigen receptor (CAR), and wherein the obtain Treg is an CAR-Treg.
Item B25: An engineered TIRC7 specific T regulatory cell (Treg), comprising a membrane bound TIRC7 specific antigen binding protein (ABP), or a TIRC7 specific antigen binding protein fragment (ABPF).
Item B26: The engineered TIRC7 specific Treg according to item B 25, wherein the TIRC7 specific ABP, or TIRC7 specific ABPF, is an ABP or ABPF as defined in any of item Bs 1 to 15.
Item B27: The engineered TIRC7 specific Treg according to any one of item Bs 25 to 26, which expresses a chimeric antigen receptor (CAR) comprising in that order an (i) extracellular domain comprising a TIRC7 specific antigen binding site, (ii) a membrane domain or membrane anchor, and (iii) an intracellular domain comprising a signalling domain.
Item B28: The engineered TIRCT7 specific Treg according to item B 25, comprising and/or capable of expressing, the TIRC7 specific CAR according to any one of item Bs 1 to 7.

The terms “of the [present] invention”, “in accordance with the invention”, “according to the invention” and the like, as used herein are intended to refer to all aspects and embodiments of the invention described and/or item Bed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, 10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

The figures show:

FIG. 1: Data on freshly isolated CD4+CD25+ TIRC7+ cells or CD4+CD25− TIRC7+ resting or activated cells from 5 subjects shows TIRC7 expression is higher in CD4+CD25+ cell subsets.

FIG. 2: TIRC7 is predominantly expressed on human (CD4+/CD25+) T cells compared to (CD4+/CD25-) T cells

FIG. 3: a) TIRC7 is predominantly expressed in CD25+CD4+ cells in mice. Flow cytometometric analysis of TIRC7 expression in CD4+ splenocytes. b) Flow cytometometric analysis of TIRC7 expression in bead-separated CD4+CD25+ or CD4+CD25− splenocytes shows higher expression of TIRC7 in resting and activated CD4+CD25+ cells. Naïve splenocyte of 12 weeks old Balb/c mice were separated via MACS beads. CD4+CD25+ cells and CD4+CD25− cells were cultured in RPMI medium supplemented with 1% BSA, 100 U/ml penicillin, 100 ug/ml streptomycin sulfate, 2 mM L-glutamine. The cells were untreated or treated with PHA (2 ug/ml) for 24 hrs. TIRC7 expression was analyzed by flow cytometry using FITC-labeled anti-TIRC7 mAb.

FIG. 4: CD4+CD25+ TIRC7+ cells inhibit CD3+ as well as CD3− splenocyte proliferation after mitogen-stimulation of splenocytes in mice compared to positive control (stim). Briefly, 12 weeks old Balb/c mouse CD4+CD25+ cells were isolated by using microbeads from naive splenocytes, and stimulated with PHA and IL2 (1:1000). Cells were cultured under 5% C02, 37° C. for 48 hrs with and without CD4+CD25+ TIRC7+ Treg cells. Proliferation was assessed by 3H-thymidine methods.

FIG. 5: The predicted secondary structure of TIRC7 protein contains seven transmembrane spanning domains (TM). Antibody binding regions according to the invention are denoted P1-P7 and can be used to generate AB synthesized according to sequences in the putative intracellular amino terminus (NH3), extracellular carboxy terminus (COOH), and the largest intracellular (IC) and extracellular (EC) loop. Preferred regions are given in the box.

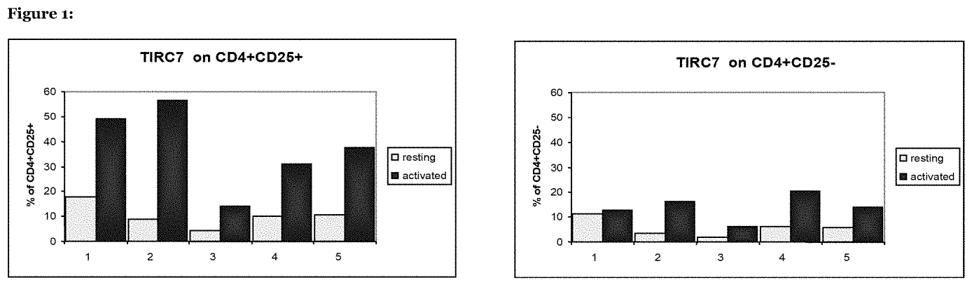

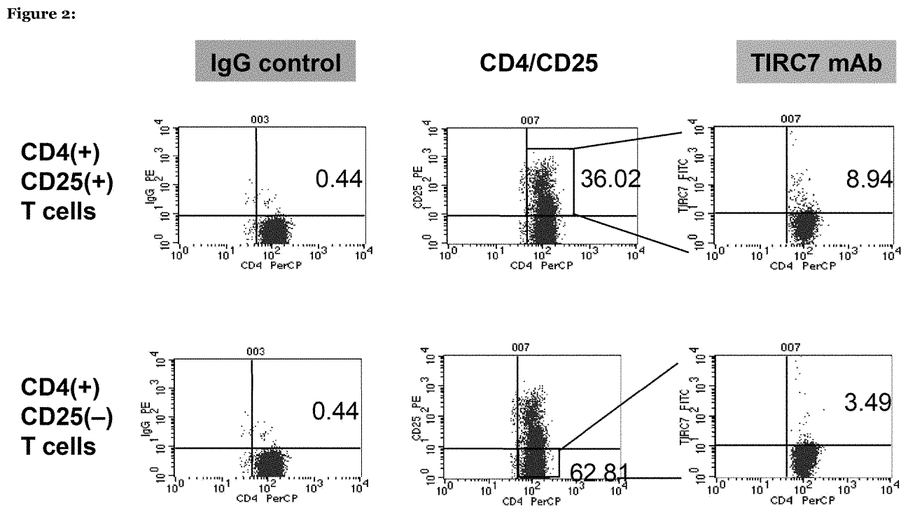

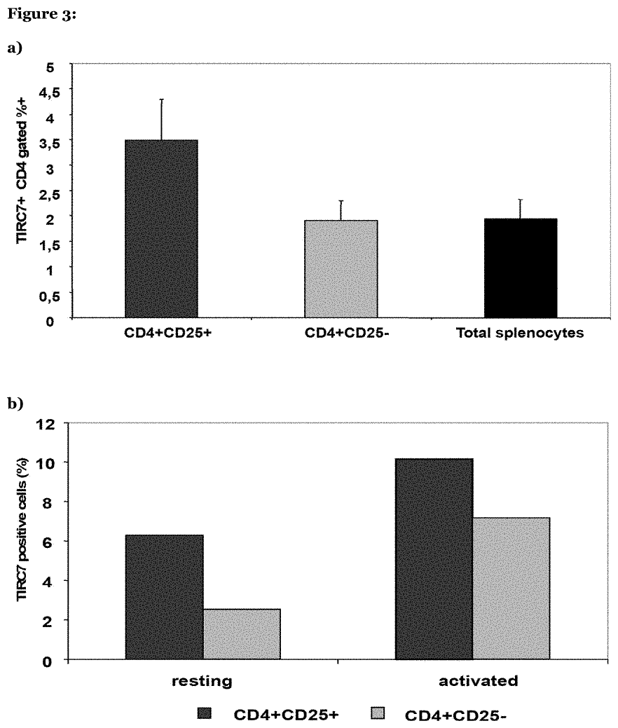

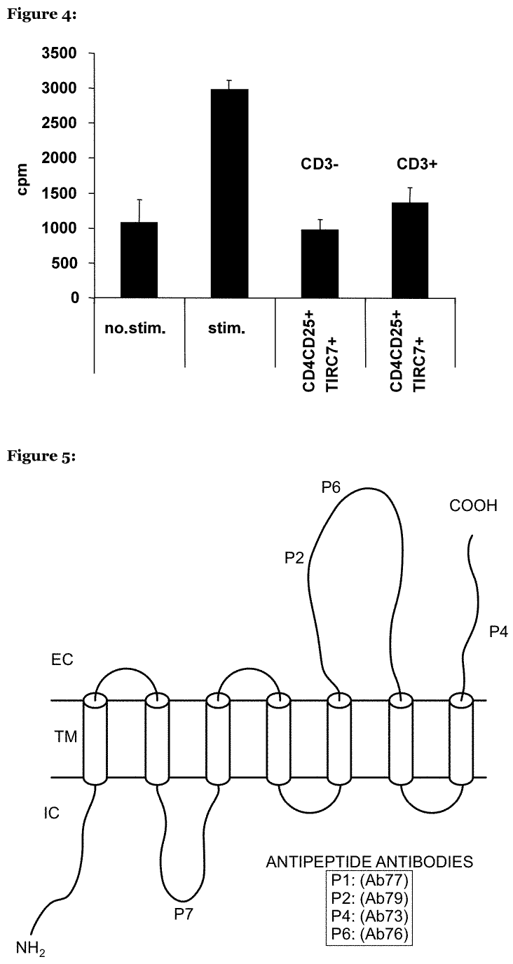

And the sequences show:

V-type proton ATPase 116 kDa subunit a isoform 3
(long isoform):
SEQ ID NO: 1
MGSMFRSEEVALVQLFLPTAAAYTCVSRLGELGLVEFRDLNASVSAFQRR
FVVDVRRCEELEKTFTFLQEEVRRAGLVLPPPKGRLPAPPPRDLLRIQEE
TERLAQELRDVRGNQQALRAQLHQLQLHAAVLRQGHEPQLAAAHTDGASE
RTPLLQAPGGPHQDLRVNFVAGAVEPHKAPALERLLWRACRGFLIASFRE
LEQPLEHPVTGEPATWMTFLISYWGEQIGQKIRKITDCFHCHVFPFLQQE
EARLGALQQLQQQSQELQEVLGETERFLSQVLGRVLQLLPPGQVQVHKMK
AVYLALNQCSVSITHKCLIAEAWCSVRDLPALQEALRDSSMEEGVSAVAH
RIPCRDMPPTLIRTNRFTASFQGIVDAYGVGRYQEVNPAPYTIITFPFLF
AVMFGDVGHGLLMFLFALAMVLAENRPAVKAAQNEIWQTFFRGRYLLLLM
GLFSIYTGFIYNECFSRATSIFPSGWSVAAMANQSGWSDAFLAQHTMLTL
DPNVTGVFLGPYPFGIDPIWSLAANHLSFLNSFKMKMSVILGVVHMAFGV
VLGVFNHVHFGQRHRLLLETLPELTFLLGLFGYLVFLVIYKWLCVWAARA
ASAPSILIHFINMFLFSHSPSNRLLYPRQEVVQATLVVLALAMVPILLLG
TPLHLLHRHRRRLRRRPADRQEENKAGLLDLPDASVNGWSSDEEKAGGLD
DEEEAELVPSEVLMHQAIHTIEFCLGCVSNTASYLRLWALSLAHAQLSEV
LWAMVMRIGLGLGREVGVAAVVLVPIFAAFAVMTVAILLVMEGLSAFLHA
LRLHWVEFQNKFYSGTGYKLSPFTFAATDD
V-type proton ATPase 116 kDa subunit a isoform 3
(short isoform - TIRC7):
SEQ ID NO: 2
MTFLISYWGEQIGQKIRKITDCFHCHVFPFLQQEEARLGALQQLQQQSQE
LQEVLGETERFLSQVLGRVLQLLPPGQVQVHKMKAVYLALNQCSVSTTHK
CLIAEAWCSVRDLPALQEALRDSSMEEGVSAVAHRIPCRDMPPTLIRTNR
FTASFQGIVDAYGVGRYQEVNPAPYTIITFPFLFAVMFGDVGHGLLMFLF
ALAMVLAENRPAVKAAQNEIWQTFFRGRYLLLLMGLFSIYTGFIYNECFS
RATSIFPSGWSVAAMANQSGWSDAFLAQHTMLTLDPNVTGVFLGPYPFGI
DPIWSLAANHLSFLNSFKMKMSVILGVVHMAFGVVLGVFNHVHFGQRHRL
LLETLPELTFLLGLFGYLVFLVIYKWLCVWAARAASAPSILIHFINMFLF
SHSPSNRLLYPRQEVVQATLVVLALAMVPILLLGTPLHLLHRHRRRLRRR
PADRQEENKAGLLDLPDASVNGWSSDEEKAGGLDDEEEAELVPSEVLMHQ
AIHTIEFCLGCVSNTASYLRLWALSLAHAQLSEVLWAMVMRIGLGLGREV
GVA

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

PBMC Isolation

Obtain a buffy coat from healthy donor from Blood Bank or other source. Add 10 ml blood/50 ml tube and dilute each tube with 20 ml PBS 1×. Layer the bottom of each tube with 10 ml Ficoll-Hypaque. Spin the tubes down at 3000 rpm (500 g) at 22° C. for 30 min. Cell count.

Isolation of Regulatory TIRC7+ T cells

Isolate PBMC from HLA-matched Donor or patient with autoimmune disease. Freeze 50×10\hat{ }6 PBMC to be used as effector cell for suppression assay. Cells are spun down at 300 g for 10 minutes and pellet re-suspended in MACS buffer. Re-suspend in 90 μl MACS buffer/10\hat{ }7 cells. Add 10 μl CD25+ TIRC7+ Microbeads/10\hat{ }7 cells, mix and incubate at 4° C. for 15 min. Wash cells. Adjust volume to 500 μl/10 8 cells. LS columns are placed in the magnetic field of a MACS separator and equilibrated with 3×3 ml of MACS buffer.

Cells are applied to the column. The unlabeled cells are collected as they pass through the column. The columns are washed 3 times. Remove column from magnet. Add 5 ml buffer and flush out cells. Cell count Treg cells.

Treg Expansion

Co-culture Tregs with CD3 & CD28+ beads in 1 mL of complete media containing IL-2 (10000 IU/mL), IL-15 (10 ng/mL) and 100 ng/mL Rapamycin. Incubate at 37° C., 95% humidity, and 5% CO₂ for 12-14 days. On Day 6, 8 and 10 take out 0.6 ml of media and replace with 0.6 ml of fresh media containing cytokines. At the end of expansion count the cells with trypan blue to determined fold expansion.

Purity

Perform phenotype analysis of the following samples to determine Treg purity and other cells contaminants using flow cytometry:

Treg Panel: CD3 PB, CD4 PercpCy5.5, CD8 APC-Cy7, CD25 PE-Cy7, CD127 Alexa647, Foxp3 PE, Live Dead Yellow

Live/Dead Staining (Use for Intracellular Staining)
I. Bring one vial of the fluorescent reactive Live/dead yellow dye (Component A) and the vial of anhydrous DMSO (Component B) to room temperature before removing the caps.
II. Add 50 μl of DMSO (Component B) to the vial of reactive dye (Component A). Mix well and visually confirm that all of the dye has dissolved.
III. Use the working solution of reactive dye as soon as possible or store at −20° C., protected from light and moisture for later use (until 2 weeks).
IV. Centrifuge cells at 1500 rpm for 5 minutes at 4° C. and discard the supernatant
V. Transfer the cells to flow tubes, count the cells to adjust the density with PBS to 0.5-1×10e6 cells in a 1 ml volume.
VI. Add 1 μl of the reconstituted fluorescent reactive dye (from step 2) to 1 ml of the cell suspension and mix well.
VII. Incubate on ice for 30 minutes, protected from light.
VIII. Add 2 ml of staining buffer and centrifuge at 1500 rpm for 5 minutes.
IX. Proceed to multicolor cell surface antigen staining

Multicolor Staining of Cell Surface Antigens
X. After Live/Dead staining, wash the cells once with 1 ml of staining buffer.
XI. Discard the supernatant, resuspend the cell pellet in 100 uL of staining buffer
XII. Properly label the tubes containing cells as mentioned in table. Add each fluorochrome-labeled antibody to the sample or FMO control tubes as mentioned.
XIII. For compensation control beads, add one drop of each positive and negative anti-mouse compensation beads to comp tubes and add 100 μl of staining buffer. Add each fluorochrome-labeled antibody in compensation tubes.
XIV. Incubate cells at 4° C. for 15 minutes, protected from light.
XV. Add 2 ml of staining buffer, centrifuge at 1500 rpm for 5 minutes, discard the supernatant and resuspend in 300 μl of staining buffer.
XVI. Analyze sample by Flow cytometry or proceed to intracellular staining

Intracellular Staining (for Samples that Require Intracellular Staining, i.e.: CD25 Purity Panel)
1. Preparation of Foxp3 Buffer before use
   Dilute Foxp3 Buffer A (lox concentrate) 1:10 with RT dsH2O
   To make a working solution of Buffer C, dilute Foxp3 Buffer B (50×) at a ratio of 1:50 (Buffer B:Buffer A)
2. After surface staining, wash cells with 2 mL staining buffer, 500 g for 5 min at 4° C.
3. Vortex pellet while adding 2 ml of 1× Foxp3 Buffer A
4. Incubate at room temperature for 10 minutes
5. Centrifuge at 500 g for 5 minutes at 4° C., decant supernatant
6. Wash cells with 2 ml of Staining Buffer
7. Centrifuge at 500 g for 5 minutes at 4° C., remove supernatant
8. While vortexing, add 500 uL of 1× Working Solution Foxp3 Buffer C
9. Incubate at room temperature for 30 minutes, repeat step 6 and 7
10. Add 50 uL of Staining Buffer followed by intracellular antibody as mentioned and vortex
11. Incubate at room temperature for 30 minutes
12. Wash as in step 6 and 7. Resuspend the cells in 250 uL of staining buffer
13. Analyze the sample on the flow cytometer

Demonstrate Suppressor Function of Expanded Tregs
Isolate effector cells CD4+ CD25−
Deplete CD25 using microbeads
Isolate CD4 T cells using T cell isolation kit from Miltenyi
Culture 5×10⁴ isolated CD4+CD25− T cells with CD3+&CD28+ beads
Add expanded Tregs at 1:2, 1:20, 1:200, 1:2000, 1:20000 (Treg:effector) to either CD3+ or CD3− population
Add expanded Tregs starting with 25000, 2500, 250, 25, 2.5 in 200 uL of culture medium at 37° C., 95% humidity, and 5% CO₂ in 96-well, round bottom plates
Culture for 6 days
Add (1 uCi) [³H] to each well during the last 16 hrs of culture
Analyze Proliferation by [³H] thymidine incorporation using gas scintillation counter

1. An in-vitro method for generating a culture of enriched, preferably human, modified regulatory T-cells (Treg) composition, the method comprising the steps of:
(i) Providing at least one Treg,
(ii) Contacting the Treg with at least one TIRC7 specific antigen binding protein (ABP), or a Tirc7 specific antigen binding protein fragment (ABPF) thereof,
(iii) Multiplying by culturing the at least one Treg in the presence of the least one TIRC7 specific ABP or ABPF to obtain the enriched modified Treg composition;
(iv) Optionally, harvesting the enriched modified Treg composition.

2. The method of claim 1, wherein step (b) comprises (i) contacting an isolated ABP or ABPF protein with the Treg, or (ii) introducing into said Treg a first genetic expression construct for the expression of the at least one TIRC7 specific ABP or ABPF, preferably wherein the method further comprises a step of expressing said first genetic expression construct in said Treg to obtain a modified Treg.

3. The method according to claim 1, wherein said Treg is a Treg isolated from a (human) patient suffering from a disorder, preferably wherein the disorder is treatable by modulation of the activity of Treg in the patient, such as a cancer or immune disorder.

4. The method according to claim 1, wherein said TIRC7 specific ABP or ABPF, specifically binds an extracellular domain, or the C-terminus of TIRC7.

5. The method of claim 4, wherein the ABP or ABPF specifically (i) binds the extracellular domain of TIRC7 between the 1^{st} and the 2^{nd} transmembrane domain, or (ii) binds the extracellular domain of TIRC7 between the 3^{st} and the 4^{th} transmembrane domain, or (iii) binds the extracellular domain of TIRC7 between the 5^{th} and the 6^{th} transmembrane domain, or (iv) binds the c-terminal region c-terminal of the 7^{th} transmembrane domain, wherein the numbering of the transmembrane domains is in the direction of N to C terminus.

6. The method of claim 1, wherein the step (b) further comprises introducing into said Treg a second genetic expression construct for the expression of a second TIRC7 specific ABP, or TIRC7 specific ABPF thereof, wherein said second TIRC7 specific ABP is different from the first TIRC7 specific ABP or ABPF, and preferably wherein the first and the second TIRC7 specific ABP or ABPF, bind to different TIRC7 epitopes.

7. The method of claim 6, wherein the first and the second TIRC7 specific ABP or ABPF, specifically bind different extracellular regions of TIRC7, preferably different regions, numbers (i) to (vi), for example wherein the first TIRC7 specific ABP or ABPF binds to the C-terminal region c-terminal of the 7^{th} transmembrane domain, and the second ABP specifically binds TIRC7 at an extracellular domain (i) to (iii).

8. The method of claim 1, wherein the step (b) further comprises introducing into said Treg at least one additional genetic expression construct for the expression of at least one further (3^{rd}, 4^{th}, 5^{th}, 6^{th} etc.) TIRC7 specific ABP or ABPF, wherein said at least one further TIRC7 specific ABP is different from the first or second TIRC7 specific ABP.

9. The method of claim 1, wherein at least one ABP or ABPF is selected from a T cell receptor, or a fragment thereof, or an antibody, or a fragment thereof.

10. The method of claim 1, wherein the ABP or ABPF is an extracellular protein, and preferably located to the cell membrane, for example, the ABP or ABPF comprises at least one transmembrane domain, or other membrane anchor.

11. The method of claim 1, wherein the ABP or ABPF comprises at least one antigen binding site derived from an antibody or T cell receptor, and preferable comprises an antigen binding fragment of an antibody such as an antibody Fab, or antibody scFv.

12. The method of claim 1, wherein the TIRC7 specific ABP or ABPF binds to an extracellular sequence of TIRC7 selected from:
(i) amino acids 210-228 of SEQ ID NO:2;
(ii) amino acids 347-355 of SEQ ID NO:2;
(iii) amino acids 438-512 of SEQ ID NO:2; or
(iv) amino acids 586-614 of SEQ ID NO:2.

13. The method of claim 1, wherein the TIRC7 specific ABP is an antibody, or antigen binding fragment thereof, selected from Neliximab or Metiliximab, or CDR grafted humanized versions thereof, or any antigen binding fragment of Neliximab or Metiliximab.

14. The method of claim 1, wherein an ABPF, or other fragments of an antibody, are preferably proteins comprising at least one antibody or TCR variable region, preferably two antibody or TCR variable regions, such as an Fab or scFv fragment.

15. The method of claim 1, wherein the TIRC7 specific ABP is a chimeric antigen receptor (CAR), and wherein the obtain Treg is a CAR-Treg.

16. An engineered TIRC7 specific T regulatory cell (Treg), comprising a membrane bound TIRC7 specific ABP or ABPF.

17. A TIRC7 specific chimeric antigen receptor (CAR), comprising an ectodomain, a transmembrane domain, and optionally an endodomain, wherein the ectodomain comprises at least one antigen binding site for binding to TIRC7 (SEQ ID NO:2).

18. The TIRC7 specific CAR of claim 17, wherein the antigen binding site for binding to TIRC7 is an antibody derived antigen binding site specific for TIRC7, preferably wherein the antibody derived binding site is derived from an anti-TIRC7 antibody which, upon binding to TIRC7, reduces T cell activity.

19. The TIRC7 specific CAR of claim 18, wherein the antibody derived antigen binding site is at least one antibody variable domain, or antigen binding fragment thereof, preferably is two antibody variable domains.

20. The TIRC7 specific CAR of claim 18, wherein the antibody derived antigen binding site specific for TIRC7 is an Fab, scFv, or antigen binding site derived from an antibody specific for TIRC7, such as Neliximab or Metiliximab, or is a human or humanized antibody versions thereof, Fab or scFv obtained by CDR grafting of the CDR regions of Neliximab or Metiliximab into human variable frameworks.

21. The TIRC7 specific CAR of claim 17, wherein the ectodomain comprises the 6 CDRs of Neliximab or comprises the 6 CDRs of Metiliximab.

22. The TIRC7 specific CAR of claim 1, wherein the transmembrane domain is a CD28 transmembrane domain, or is a membrane anchor.

23. The TIRC7 specific CAR of claim 17, wherein the endodomain comprises an immune signalling domain, preferably the endodomain comprises a CD3-zeta endodomain with 3 ITAMs.

24. A nucleic acid molecule comprising a sequence encoding the TIRC7 specific CAR according to claim 1.

25. A recombinant cell, preferably a regulatory T cell (Treg) comprising the TIRC7 specific CAR or a nucleic acid according to claim 1.


SEQUENCE LISTING (2 sequences)

SEQ ID NO: 1 - protein, 830 aa, Homo sapiens
MGSMFRSEEVALVQLFLPTAAAYTCVSRLGELGLVEFRDLNASVSAFQRRFVVDVRRCEELEKTFTFLQEEVRRAGLVLPPPKGRLPAPPPRDLLRIQEETERLAQELRDVRGNQQALRAQLHQLQLHAAVLRQGHEPQLAAAHTDGASERTPLLQAPGGPHQDLRVNFVAGAVEPHKAPALERLLWRACRGFLIASFRELEQPLEHPVTGEPATWMTFLISYWGEQIGQKIRKITDCFHCHVFPFLQQEEARLGALQQLQQQSQELQEVLGETERFLSQVLGRVLQLLPPGQVQVHKMKAVYLALNQCSVSTTHKCLIAEAWCSVRDLPALQEALRDSSMEEGVSAVAHRIPCRDMPPTLIRTNRFTASFQGIVDAYGVGRYQEVNPAPYTIITFPFLFAVMFGDVGHGLLMFLFALAMVLAENRPAVKAAQNEIWQTFFRGRYLLLLMGLFSIYTGFIYNECFSRATSIFPSGWSVAAMANQSGWSDAFLAQHTMLTLDPNVTGVFLGPYPFGIDPIWSLAANHLSFLNSFKMKMSVILGVVHMAFGVVLGVFNHVHFGQRHRLLLETLPELTFLLGLFGYLVFLVIYKWLCVWAARAASAPSILIHFINMFLFSHSPSNRLLYPRQEVVQATLVVLALAMVPILLLGTPLHLLHRHRRRLRRRPADRQEENKAGLLDLPDASVNGWSSDEEKAGGLDDEEEAELVPSEVLMHQAIHTIEFCLGCVSNTASYLRLWALSLAHAQLSEVLWAMVMRIGLGLGREVGVAAVVLVPIFAAFAVMTVAILLVMEGLSAFLHALRLHWVEFQNKFYSGTGYKLSPFTFAATDD

SEQ ID NO: 2 - protein, 614 aa, Homo sapiens
MTFLISYWGEQIGQKIRKITDCFHCHVFPFLQQEEARLGALQQLQQQSQELQEVLGETERFLSQVLGRVLQLLPPGQVQVHKMKAVYLALNQCSVSTTHKCLIAEAWCSVRDLPALQEALRDSSMEEGVSAVAHRIPCRDMPPTLIRTNRFTASFQGIVDAYGVGRYQEVNPAPYTIITFPFLFAVMFGDVGHGLLMFLFALAMVLAENRPAVKAAQNEIWQTFFRGRYLLLLMGLFSIYTGFIYNECFSRATSIFPSGWSVAAMANQSGWSDAFLAQHTMLTLDPNVTGVFLGPYPFGIDPIWSLAANHLSFLNSFKMKMSVILGVVHMAFGVVLGVFNHVHFGQRHRLLLETLPELTFLLGLFGYLVFLVIYKWLCVWAARAASAPSILIHFINMFLFSHSPSNRLLYPRQEVVQATLVVLALAMVPILLLGTPLHLLHRHRRRLRRRPADRQEENKAGLLDLPDASVNGWSSDEEKAGGLDDEEEAELVPSEVLMHQAIHTIEFCLGCVSNTASYLRLWALSLAHAQLSEVLWAMVMRIGLGLGREVGVAAVVLVPIFAAFAVMTVAILLVMEGLSAFLHALRLHWVEFQNKFYSGTGYKLSPFTFAATDD